(19) United States
(12) Patent Application Publication
(10) Pub. No.: US 2008/0113936 A1
(43) Pub. Date: May 15, 2008

(54) PROCESS FOR THE PREPARATION OF ADSORBATES OF VALSARTAN AND/OR ITS SOLVATES OR HYDRATES

(76) Inventor: KLAUS GLAENZER, Hamburg (DE)

Correspondence Address: J.C. Patents;Suite 250, 4 Venture, Irvine, CA, 92618, US

(21) Appl. No.: 12/014,729

(22) Filed: Jan. 15, 2008

Related U.S. Application Data
(62) Division of application No. 11/107,070, filed on Apr. 15, 2005.

(30) Foreign Application Priority Data
Apr. 15, 2004 (EP) 04 101 545.4

(51) Int. Cl. A61K 31/724; A61K 31/717; A61K 31/4184

(52) U.S. Cl. 514/57; 514/58; 514/381

(57) ABSTRACT

The present invention relates to a process for the preparation of adsorbates of valsartan and/or its solvates or hydrates wherein one starts with a solution of valsartan or of one of its pharmaceutically acceptable salts and/or their solvates or hydrates in at least one organic solvent where the total water content of the solvent is not higher than 15% by volume, preferably not higher than 5% by volume, disperses therein an adsorbing material selected from the group consisting of celluloses, cellulose derivatives, polyols, sugars, sugar derivatives, maltodextins, cyclodextrins, starches, polydextroses, or their mixtures, and removes the solvent. The invention further relates to valsartan adsorbates that can be prepared with the processes precited, as well as pharmaceutical formulations for the preparation of which the valsartan adsorbates are employed.

### CROSS-REFERENCE TO RELATED APPLICATION

This application claims benefit of priority of European Patent Application No. 04 101 545.4 filed on Apr. 15, 2004, which is incorporated herein by reference in its entirety for all purposes.

### BACKGROUND OF THE INVENTION

1. Field of the Invention

The present invention relates to a novel process for the preparation of adsorbates of valsartan and/or its solvates or hydrates. In a particularly preferred embodiment, the adsorbates according to the present invention contain the active ingredient or one of its pharmaceutically acceptable salts and/or their solvates or hydrates in a finely divided, amorphous form. The invention further relates to valsartan adsorbates that are obtainable by said process, as well as pharmaceutical formulations prepared while employing said valsartan adsorbates. Preferred drug formulations according to the invention are tablets, capsules, pellets, and granules prepared with the usual, pharmaceutically acceptable adjuvants in ways known per se. Particularly preferred according to the invention are tablets rapidly releasing the active ingredient that are prepared by direct compressing of the valsartan adsorbates according to the present invention.

2. Description of Related Art

The active ingredient having the chemical name of (S)—N-(1-carboxy-2-methylprop-1-yl)-N-pentanoyl-N-[2′-(1H-tetrazol-5-yl)biphenyl-4-yl-methylamine also known by the INN valsartan can be represented by the following structural formula:

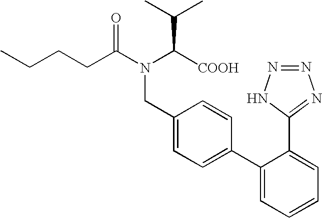

Valsartan belongs to the class of non-peptide angiotensin-II receptor antagonists having a very high selectivity for the AT₁ receptors. With a common daily dose of 80 to 160 mg, valsartan is used as a single-substance preparation (mono-preparation) or in combination with the diuretic hydrochlorothiazide for the treatment of cardiovascular diseases. As AT₁ receptor antagonist, valsartan more particularly inhibits the blood pressure rise caused by angiotensin II, suppresses angiotensin-II-induced aldosteron secretion, and lowers angiotensin-II-induced liquid uptake (see, for instance, in Allgemeine und spezielle Pharmakologie und Toxikologie, W. Forth, D. Henschler, W. Rummel, U. Förstermann, and K. Starke, editors, 8^{th} edition, Urban & Fischer, Munich/Jena, 2001).

Valsartan, its pharmaceutically acceptable salts and processes for its preparation are described in EP 0 443 983 B1, Example 16. The preparation of valsartan salts is also described in WO 02/06253, for instance.

It is known from the prior art that valsartan is not only present as an amorphous solid but can exist as well in several crystalline or partly crystalline forms or as a mixture of various polymorphs with amorphous material (cp. WO 02/06253; WO 03/089417). The data reported in different patent documents for the melting points of valsartan polymorphs differ considerably between reported values ranging from 80° C. to 117° C. In WO 03/089417, a valsartan I form with melting points between 80° C. and 91° C. and a valsartan II form with melting points between 91° C. and 102° C. are described. The valsartan prepared along different synthetic routes reported in EP 0 443 963 B1 had melting points between 105° C. and 115° C. (D); between 105° C. and 115° C. (C); between 116° C. and 117° C. (B); and between 105° C. and 115° C. (A), without referring to specific polymorphs. In the 13^{th} edition of the Merck Index, a melting point of 116° C. to 117° C. is reported for valsartan crystals from diisopropyl ether. It must be concluded, therefore, that the preparation of a definite polymorphic form very strongly depends on process parameters or solvents, and different products will be obtained following slight changes in these parameters. This leads to the need for a very demanding process and quality control, since a polymorph that can be reproducibly prepared and is neatly defined is unconditionally needed, both in order to satisfy regulatory requirements and in order to secure constant quality of the drug and thus guarantee the pharmaceutical properties and safe consumption by patients. The use of amorphous valsartan is one possibility to solve this problem and arrive at a more advantageous isolation procedure. However, a process according to which a completely amorphous active ingredient could be prepared in a reproducible and safe fashion is not known so far. According to experimental experience gathered by the inventor of the present application, in the precipitation of the amorphous material often heterogeneous products appear, that is, mixtures with crystalline and amorphous fractions, which means that the precipitation must be repeated. Thus, apart from inevitable losses of substance the preparation requires additional effort. However, no experience is available so far as to the stability of the amorphous substance, or more particularly as to a phase change that cannot be ruled out to occur during storage or further processing.

Beyond that, the manufacture of pharmaceutical preparations containing amorphous active ingredients is a general problem necessitating technical efforts which in part are considerable. The simple solution of a direct compressing of powder mixtures with amorphous active ingredients—if at all possible with the high percentage of active ingredient in the drug that is required in the present case of valsartan—contains the risk of separation processes leading to an inhomogeneous distribution of the active ingredient that is inacceptable pharmaceutically. Dry and wet granulation procedures must for instance be performed in prior process steps in order to avoid fluctuations in the percentage of active ingredients and keep them within the limits admissible according to the pharmacopeia, on one hand, and to obtain an active substance that can be processed, on the other hand. Granules that can be pressed are finally obtained after drying, comminution, and classifying. It is known from WO 00/38676 and EP 1 140 071 B1, respectively, that even in the case of crystalline valsartan, a dry granulating process must be employed when processing the active substance to drug formulations.

Those skilled in the art know the corresponding procedures, which are technically demanding and work-intensive (see, for instance, in Die Tablette, W. A. Ritschel and A. Bauer-Brandl, 2^{nd} ed., ECV-Editio Cantor publishers, 2002) and will be able to tackle the problem in the case of crystalline valsartan, only with a large technical operating effort. Known processes for the preparation of pharmaceutical formulations containing amorphous valsartan require at least a comparable technical effort and are time and cost intensive, if they can at all be realized.

### BRIEF SUMMARY OF THE INVENTION

Therefore, object of the present invention is to develop a simple and economical process for the preparation of valsartan powder systems that can be used immediately for manufacturing pharmaceutical formulations, this process not being restricted, however, to a particularly preferred morphology of the active ingredient, and avoiding the disadvantages discussed above.

### BRIEF DESCRIPTION OF THE DRAWINGS

FIG. 1 shows powder X-ray diffraction patterns of a valsartan-lactose adsorbate according to the present invention from ethanol (in a ratio of 1:1) (upper curve) and, for comparison, of lactose alone (lower curve).

FIG. 2 shows powder X-ray diffraction patterns of a valsartan-mannitol adsorbate according to the present invention from ethanol (in a ratio of 1:1) (upper curve) and, for comparison, of mannitol alone (lower curve).

FIG. 3 shows a powder X-ray diffraction patterns of a valsartan-mannitol adsorbate according to the present invention from ethyl acetate (in a ratio of 1:1) (upper curve) and, for comparison, of mannitol alone (lower curve).

FIG. 4 shows a powder X-ray diffraction patterns of valsartan (crystallized from diisopropyl ether), revealing a heterogeneous mixture of crystalline and amorphous phases.

### DETAILED DESCRIPTION OF THE INVENTION

Therefore, the present invention relates to a process for the preparation of adsorbates of valsartan and/or of its solvates or hydrates according to which one starts from a solution of valsartan or one of its pharmaceutically acceptable salts and/or their solvates or hydrates in at least one organic solvent, the active ingredient being dispersed in the organic solvent, dissolves the adsorbing material in it, and removes the solvent, which can more particularly be achieved by drying. According to the invention, the total water content of the solvent is not higher than 15% by volume, preferably not higher than 5% by volume. In a preferred embodiment of the invention, said valsartan adsorbates contain the active ingredient or one of its pharmaceutically acceptable salts and/or their solvates or hydrates in a finely divided, amorphous form. The amorphous valsartan or one of its salts can be present, both in the anhydrous formulation and in the formulation of solvates or hydrates. The invention further relates to adsorbates of valsartan and/or pharmaceutically acceptable salts of valsartan and/or their corresponding solvates or hydrates that are obtainable by said process.

Typically, pharmaceutically acceptable salts of valsartan with bases are base addition salts with metal salts such as for instance alkali or alkaline-earth metal salts, typically sodium, potassium, calcium, or magnesium salts or salts with ammonia or organic amines such as morpholine, thiomorpholine, piperidine, pyrrolidine, mono, di or lower triaalkylamines, typically ethylamine, tert-butylamine, diethylamine, diisopropylamine, dibutylamine, triethylamine, tributylamine or dimethylpropylamine. Addition salts of valsartan with mono, di, or trihydroxy lower-alkylamines, typically mono, di, or triethanolamine, are possible as well. The corresponding inner salts can also be used.

The invention further relates to pharmaceutical formulations containing these novel valsartan adsorbates. Where applicable, the pharmaceutical formulations contain further adjuvants and can be converted to the desired drug delivery formulation. Tablets produced by direct compressing which rapidly release the active ingredient are particularly preferred.

The pharmaceutical formulations according to the invention which contain valsartan adsorbates as the AT₁ receptor antagonist can be employed to treat diseases that can for instance be described as follows:
(a) Hypertension, cardiac insufficiency, renal failure, particularly chronic renal failure, restenosis after percutaneous transluminal angioplasty, and restenosis after coronary arterial bypass surgery,
(b) arteriosclerosis, insulin resistance and syndrome X, diabetes mellitus type 2, obesity, nephropathy, renal failure, for instance chronic renal failure, hypothyreosis, the condition after cardiac infarction, coronary cardiopathy, age-related hypertension, familial dyslipidemic hypertension, all the precited diseases in connection with or without hypertension,
(c) endothelial dysfunction in connection with or without hypertension,
(d) hyperlipidemia, hyperlipoproteinemia, arteriosclerosis and hypercholesterinemia,
(e) glaucoma.

Organic solvents in which the active pharmaceutical ingredient will be dissolved are suitable for the process according to the invention, for the preparation of valsartan adsorbates. The organic solvents are more particularly selected from the group of lower alkanols with one to four carbon atoms, the group of ethers, the group of esters, the group of aliphatic ketones, and the group of halogenated hydrocarbons, as well as mixtures of said solvents. Methanol, ethanol, isopropanol, n-propanol, acetone and other solvents such as ethyl acetate, methyl ethyl ketone, diisopropyl ether, MTBE (methyl tert-butyl ether), dichloromethane, petrol ether, acetone, hexane, an acetone/water mixture, an ethyl acetate/hexane mixture, a dichloromethane/ethyl acetate mixture, as well as further mixtures of said solvents are particularly preferred.

According to the present invention, those pharmaceutically acceptable adjuvants are used as adsorbing materials which are appropriate for a rapid release of the active ingredient, such as celluloses and cellulose derivatives, more particularly lactose, maltodextrin, starches, cyclodextrins, polydextroses or mixtures of said substances. Microcrystalline cellulose, lactose, and mannitol are preferred according to the invention. For an improvement of the flow properties, additives containing silica or titania can be used.

The ratio of pharmaceutical active ingredient to adsorbing material according to the invention is in the range from 1:0.1 to 1:10, a range from 1:0.5 to 1:2 being particularly preferred.

All common pharmaceutical adjuvants can be used to prepare the pharmaceutical formulations, where tablets are more particularly preferred. As fillers, for example celluloses and cellulose derivatives (for instance microcrystalline cellulose, native cellulose, hydroxypropyl cellulose, hydroxypropyl methyl cellulose, methyl cellulose), sugars (for instance lactose, fructose, saccharose, glucose, maltose), sugar alcohols (for instance lactitol, mannitol, sorbitol, xylitol), inorganic fillers (for instance calcium phosphates and calcium sulfates), and starches (for instance corn starch, potato starch, wheat starch, dextrins, pregelatinized starches) can be used. Beyond that, all other adjuvants known to those skilled in the art from their basic galenic knowledge, such as lubricants, disintegration aids, wetting agents, agents to improve the flow behavior, other additives, stabilizers, as well as flavors, pigments, and dyes, can be used to prepare the drug formulations according to the invention (those skilled in the art can take the corresponding information, for instance, from: Die Tablette, W. A. Ritschel and A. Bauer-Brandl, 2^{nd} ed., ECV-Editio Cantor publishers, 2002).

The portion of binders in the complete mixture of the drug preparation is preferably between 0 and 20% (mass/mass), the fraction of fillers and adjuvants in the complete mixture is 2 to 80% by weight, preferably 5 to 25% by weight.

The process according to the invention yields surprisingly stable, homogeneous adsorbates of valsartan free from portions of crystalline active agent. These valsartan adsorbates are used as active pharmaceutical ingredient in the preparations according to the invention.

Within this invention, the respective hydrates, solvates, or salts of valsartan which may originate more particularly during preparation of the active ingredient at the end of the synthetic route in the solution, can also be employed, so that isolation of the pure active ingredient can be avoided.

According to the invention, a process has now been found which, starting from a solution of valsartan in an organic solvent, leads to adsorbates of the active ingredient that can be processed directly to the drug formulation.

In an embodiment of the invention, the solution of valsartan active ingredient can on principle be prepared by dissolving the active ingredient or a salt in a suitable organic solvent; though it is more advantageous to directly use a solution of the active ingredient resulting anyhow during synthesis, without isolation of the valsartan.

Valsartan can for instance be prepared according to EP 0 443 983 B1, but omitting the recrystallization steps involving, e.g., a dissolution in diisopropyl ether or ethyl acetate. Instead, the adsorbing material is dispersed in the solution of the active ingredient, and later the solvent is removed by drying. The kind of organic solvent used then results, in any given case, from the final step of synthesis in the process chosen for preparing the active ingredient.

To this organic solution of the active ingredient, a pharmaceutically acceptable adjuvant that is insoluble or poorly soluble in it is added as the adsorbing material, well wetted, and immediately thereafter the solvent is removed by drying. The drying process can be promoted by temperature, applying a vacuum, sublimation drying for instance, or also by spray drying. Advantageously, it is conducted in such a way that appropriate mechanical action (e.g., rotating, tumbling, or stirring motion) yields a uniform distribution. The solvent can be recovered by working in a closed system, and reused for a subsequent process. According to the invention, a precipitation and isolation of the valsartan is omitted. Adsorbates prepared by the process described can be employed directly in further processing to drug formulations such as tablets, capsules, pellets, or granules, preferably in further processing by a direct compressing process.

Optionally, the adsorbates or drug formulations thus obtained can be further provided with coatings of pharmaceutical polymethacrylates such as Eudragit® films, methyl celluloses, ethyl celluloses, hydroxypropyl methyl celluloses, cellulose acetate phthalates and/or shellac in order to fill a specific application, e.g., controlled release of the active ingredient and/or taste masking. Those skilled in the art of pharmaceutics have sufficient technical possibilities to do this.

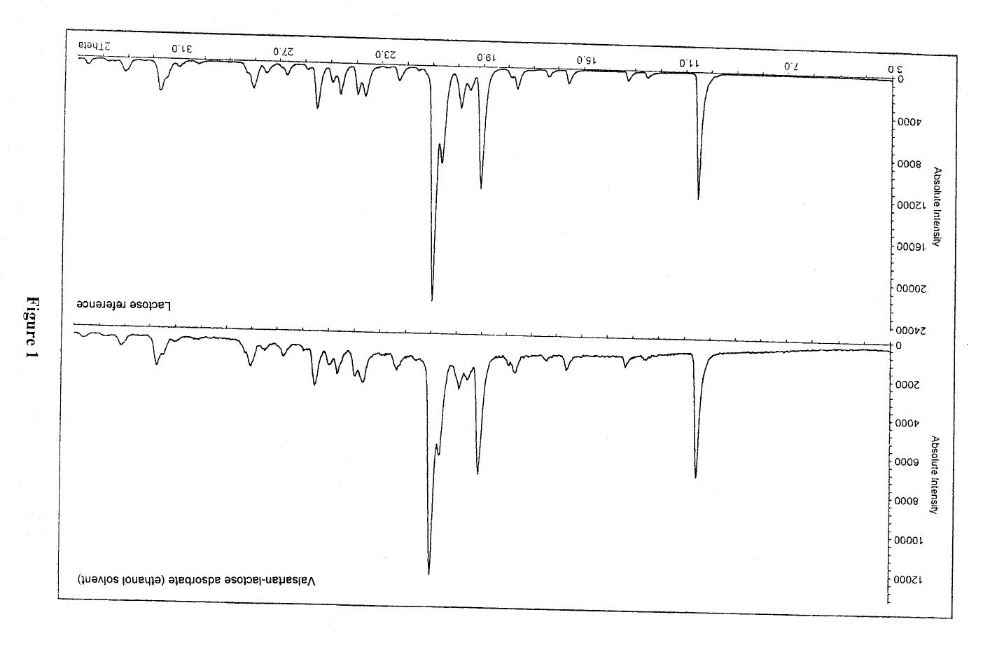

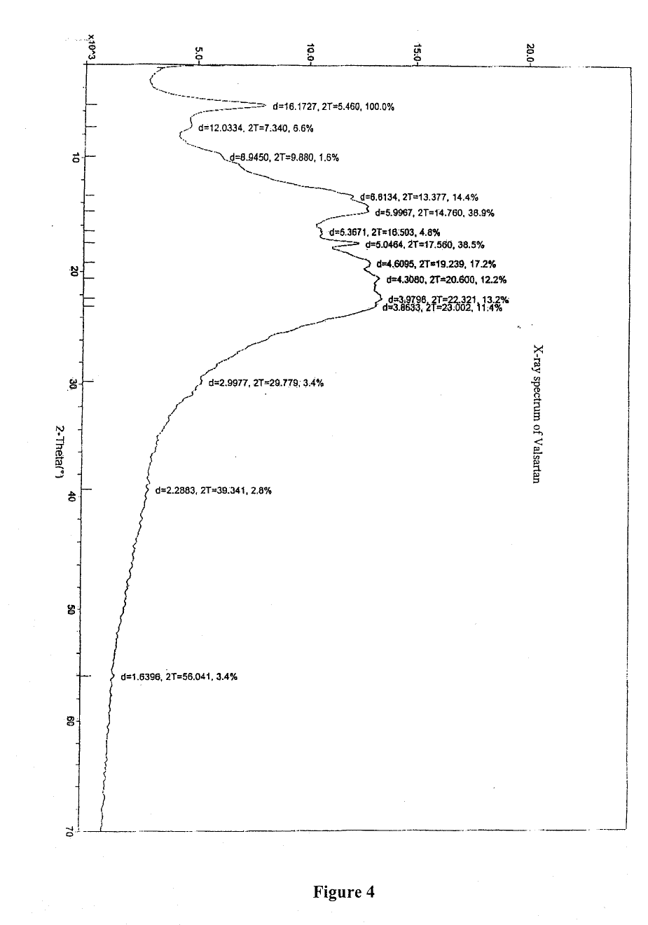

It has been found surprisingly that adsorbates prepared by the process according to the invention contain the active ingredient in the homogeneous distribution required for drugs, and release it without limitations. The adsorbates prepared by this process bind the active ingredient in such a way that that crystal structures typical of the active ingredient are not developed. This could be demonstrated by X-ray diffraction (see FIGS. 1 to 3). For comparison, FIG. 4 shows a powder X-ray diffraction pattern with a crystal structure typical of the substance.

The characteristics mentioned are retained as well, more particularly, when the valsartan adsorbates are processed to drug formulations, e.g., tablets. Moreover, this direct compressing does not entail any change in morphology or in the content of by-products or decomposition products (=sum of all impurities) in the route from active ingredient to drug formulation (tablet).

The invention will now be explained more closely by the following examples and figures, without however limiting the invention thereto.

### EXAMPLES 1 TO 3

### Methods of Analysis Used

1. HPLC method for determining the content of active ingredient or sum of all contaminants according to USP 27—In-Process Revision—Pharmacopeial Forum, Vol. 29 [November-December 2003]
2. Release of active ingredient (dissolution test) according to USP 27—In-Process Revision—Pharmacopeial Forum, Vol. 29 [November-December 2003] (required: at least 80% released after 30 min)

3. The powder X-ray diffraction patterns were recorded as follows:

| | |
|---|---|
| Instrument: | STADI P transmission diffractometer |
| | Cu Ka₁ radiation (1 = 1.54056 Å), U = 40 kV, |
| | I = 30 mA |
| | Secondary beam monochromator (flat, graphite) |
| Detector: | Scintillation counter |
| Aperture: | 2 × 8 mm; 0.7 mm; 0.35 mm |
| Linear PSD: | 2 θ = 2° to 35°, 5 s/0.04° in steps |
| Sample: | Powder, reflection mode |

### EXAMPLE 1

### Valsartan-Lactose Adsorbate

To a solution of heterogeneous valsartan (0.05 g/ml) in ethanol, 0.05 g/ml lactose (Lactopress®, anhydrous) are added and uniformly dispersed. The solvent then is dried off with continuous agitation in a vacuum (rotating vaporizer) at 25° C. In the end the mixture is post-dried for a short time at 35° C. to remove residual solvent.

| | |
|---|---|
| Theoretical content of active ingredient in the adsorbate: | 50% |
| X-ray diffraction pattern: | see FIG. 1 |

| Active ingredient | Adsorbate | 175-mg tablet |
|---|---|---|
| content by HPLC | (% valsartan) | (mg valsartan) |
| Sample No. 1 | 50.1 | 80.1 |
| Sample No. 2 | 50.2 | 80.0 |

From the adsorbate, valsartan tablets (final weight about 175 mg) were made by direct compressing in the following composition:

| | |
|---|---|
| Valsartan-lactose adsorbate corresponding to 80 mg valsartan | 160 mg |
| Adjuvants (croscarmellose sodium, sodium lauryl sulfate, | |
| silica, magnesium stearate) in the usual amounts | 15 mg |

The amounts of further adjuvants used are known to those skilled in the art from their basic knowledge, and can be taken from standard references for tablet formulation, for instance from Ritschel et al., Die Tablette, Editio Cantor, Aulendorf, 2^{nd} ed., 2002.

Properties of the mixture that is ready to be pressed, and of the tablets:

| | |
|---|---|
| Compressibility | satisfactory to good |
| and flowability: | |
| Mean hardness: | 92 N |
| Abrasion: | 0.2% (determined according to Ph. Eur.) |
| Release: | 90% after 30 min |
| Content: | see Table |
| Sum of all impurities: | Granules: 0.08%; tablet: 0.08% |

The tablets thus obtained can be provided with a coating.

### EXAMPLE 2

### Valsartan-Mannitol Adsorbate

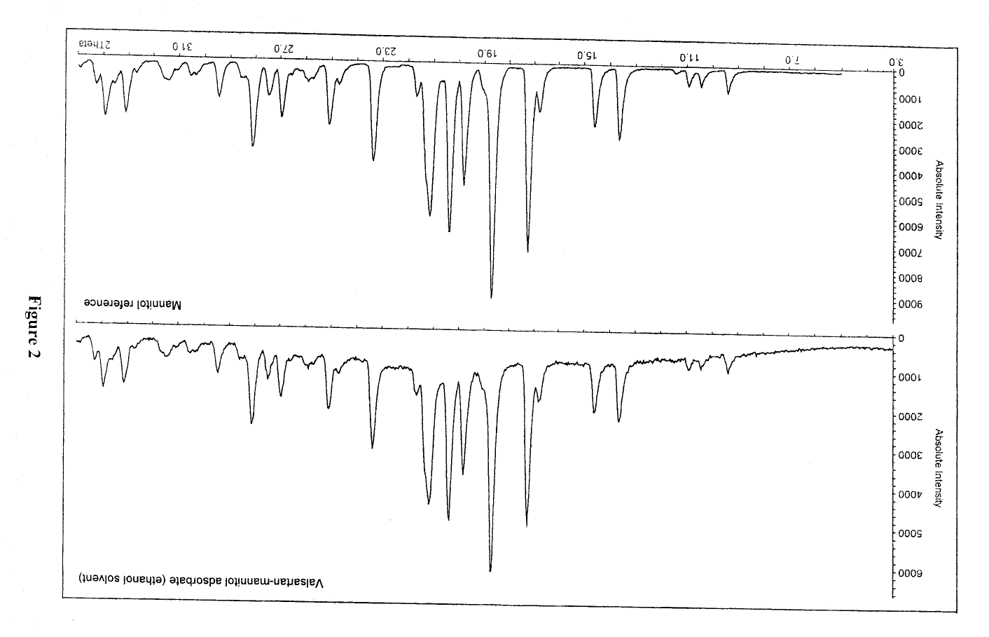

To a solution of heterogeneous valsartan (0.05 g/ml) in ethanol, 0.05 g/ml mannitol (Mannogem®) are added and uniformly dispersed. The solvent then is dried off with continuous agitation in a vacuum (rotating vaporizer) at 25° C. In the end the mixture is post-dried for a short time at 35° C. to remove residual solvent.

| | |
|---|---|
| Theoretical content of active ingredient in the adsorbate: | 50% |
| X-ray diffraction pattern: | see FIG. 2 |

| Active ingredient | Adsorbate | 175-mg tablet |
|---|---|---|
| content by HPLC | (% valsartan) | (mg valsartan) |
| Sample No. 1 | 50.1 | 79.9 |
| Sample No. 2 | 49.9 | 80.2 |

From the adsorbate, valsartan tablets (final weight about 175 mg) were made by direct compressing in the following composition:

| | |
|---|---|
| Valsartan-mannitol adsorbate corresponding to 80 mg valsartan | 160 mg |
| Adjuvants (as in example No. 1) | 15 mg |

Properties of the mixture that is ready to be pressed, and of the tablets:

| | |
|---|---|
| Compressibility | satisfactory to good |
| and flowability: | |
| Mean hardness: | 93 N |
| Abrasion: | 0.2% (determined according to Ph. Eur.) |
| Release: | 96% after 30 min |
| Content: | see Table |
| Sum of all impurities: | Granules: 0.07%; tablet: 0.07% |

The tablets thus obtained can be provided with a coating.

### EXAMPLE 3

### Valsartan-Mannitol Adsorbate

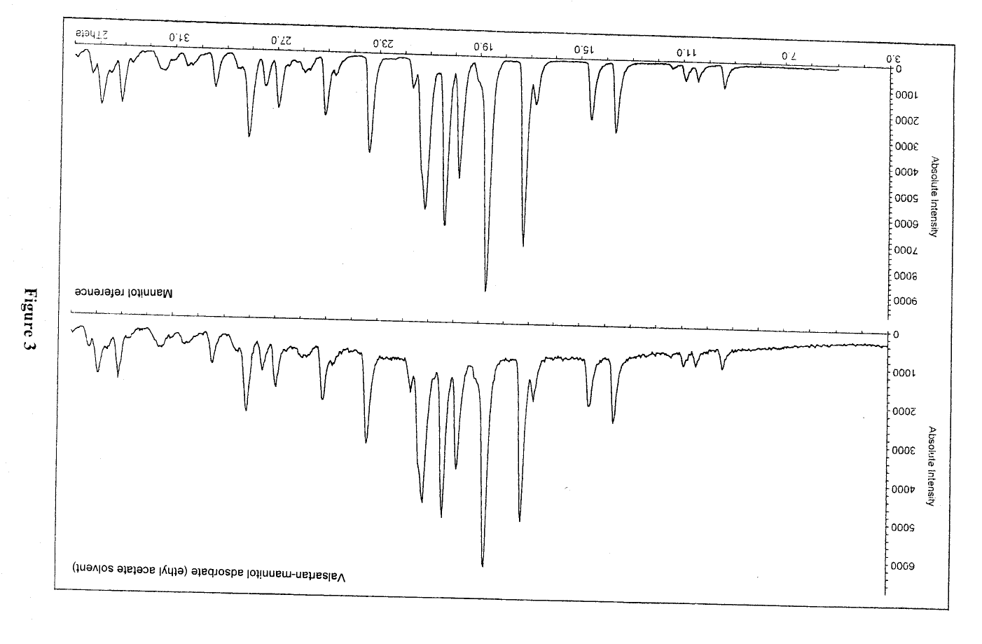

To a solution of heterogeneous valsartan (0.05 g/ml) in ethyl acetate, 0.05 g/ml mannitol (Mannogem®) are added and uniformly dispersed. The solvent then is dried off with continuous agitation in a vacuum (rotating vaporizer) at 25° C. In the end the mixture is post-dried for a short time at 35° C. to remove residual solvent.

| | |
|---|---|
| Theoretical content of active ingredient in the adsorbate: | 50% |
| X-ray diffraction pattern: | see FIG. 3 |

| Active ingredient | Adsorbate | 350-mg tablet |
|---|---|---|
| content by HPLC | (% valsartan) | (mg valsartan) |
| Sample No. 1 | 49.8 | 161.4 |
| Sample No. 2 | 51.0 | 160.9 |

From the adsorbate, valsartan tablets (final weight about 350 mg) were made by direct compressing in the following composition:

| | |
|---|---|
| Valsartan-mannitol adsorbate corresponding to 160 mg valsartan | 320 mg |
| Adjuvants (as in example No. 1) | 30 mg |

Properties of the mixture that is ready to be pressed, and of the tablets:

| | |
|---|---|
| Compressibility | satisfactory to good |
| and flowability: | |
| Mean hardness: | 112 N |
| Abrasion: | 0.2% (determined according to Ph. Eur.) |
| Release: | 91% after 30 min |
| Content: | see Table |
| Sum of all impurities: | Granules: 0.04%; tablet: 0.04% |

The tablets thus obtained can be provided with a coating.

1. A process for the preparation of adsorbates of valsartan and/or of its solvates or hydrates, wherein one starts from a solution of valsartan or of one of its pharmaceutically acceptable salts and/or their solvates or hydrates in at least one organic solvent with a total water content of the solvent of no more than 15% by volume, preferably of no more than 5% by volume, disperses in it an adsorbing material selected from the group consisting of celluloses, cellulose derivatives, polyols, sugars, sugar derivatives, maltodextrins, cyclodextrins, starches, polydextroses, or mixtures thereof, and removes the solvent.

2. The process according to claim 1, wherein the adsorbates contain the active ingredient or a pharmaceutically acceptable salt of it, or its solvates or hydrates, in a finely divided, amorphous form.

3. The process according to claim 2, wherein a ratio of active ingredient to adsorbing material in the range from 1:0.1 to 1:10, more particularly in the range from 1:0.5 to 1:2 is set.

4. The process according to claim 1, wherein a ratio of active ingredient to adsorbing material in the range from 1:0.1 to 1:10, more particularly in the range from 1:0.5 to 1:2 is set.

5. The process according to claim 1, wherein organic solvents with a total water fraction of no more than 15% by volume, preferably no more than 5% by volume, are used alone or in mixtures as the solvent, the organic solvents being selected from the group of lower alkanols with one to four carbon atoms, the group of ethers, the group of esters, the group of aliphatic ketones, and the group of halogenated hydrocarbons and their mixtures.

6. The process according to claim 5, wherein a solvent from the group consisting of methanol, ethanol, isopropanol, n-propanol, acetone, ethyl acetate, methyl ethyl ketone, methyl tert-butyl ether, dichloromethane, petrol ether, hexane, an acetone-water mixture, an ethyl acetate-hexane mixture, a mixture of dichloromethane and ethyl acetate as well as further mixtures of the aforementioned solvents is used as a solvent.

7. The process according to claim 1, wherein a solution of the active ingredient is used which is obtained in the course of valsartan synthesis.

8-15. (canceled)
